(19) United States
(12) Patent Application Publication
(10) Pub. No.: US 2008/0077218 A1
(43) Pub. Date: Mar. 27, 2008

(54) INJECTION OF THERAPEUTIC INTO POROUS REGIONS OF A MEDICAL DEVICE

(75) Inventors: Dave McMorrow, Fort Lorenzo (IE); Tim O'Connor, Claregalway (IE); Aiden Flanagan, Kilcolgan (IE); Anthony Malone, Oranmore (IE)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 11/856,994

(22) Filed: Sep. 18, 2007

Related U.S. Application Data
(60) Provisional application No. 60/846,731, filed on Sep. 25, 2006.

(51) Int. Cl. A61N 1/00; A61F 2/06

(52) U.S. Cl. 607/120; 623/1.42

(57) ABSTRACT

The present invention is directed to methods, processes, and systems for selectively driving therapeutic into at least a portion of a porous matrix of a medical implant. Under methods and processes of the invention, a medical implant may be provided having at least a portion thereof comprising a porous matrix. An injector in fluid communication with a fluid source deliver therapeutic within the porous matrix. The porous matrix may be configured to control the elution rate.

### RELATED APPLICATIONS

This application claims benefit of U.S. Provisional Application No. 60/846,731, filed Sep. 25, 2006, which is incorporated herein in its entirety.

### TECHNICAL FIELD

The present invention generally relates to injecting therapeutic into porous regions of a medical device. More specifically, the present invention relates to methods, devices, and systems that inject therapeutic from an injector into porous regions of medical devices, such as implantable stents.

### BACKGROUND

The positioning and deployment of medical devices within a target site of a patient is a common, often repeated procedure of contemporary medicine. These devices, which may be implantable stents and other devices that may be deployed for short or sustained periods of time, may be used for many medical purposes. These can include the reinforcement of recently re-enlarged lumens, the replacement of ruptured vessels, and the treatment of disease, such as vascular disease by local pharmacotherapy, i.e., delivering therapeutic drug doses to target tissues while minimizing systemic side effects. The targeted delivery areas may include body lumens such as the coronary vasculature, esophagus, trachea, colon, biliary tract, urinary tract, prostate, brain, and the like.

Coatings may be applied to the surfaces of these medical devices to increase their effectiveness. These coatings may provide a number of benefits including reducing the trauma suffered during the insertion procedure, facilitating the acceptance of the medical device into the target site, and improving the post-procedure effectiveness of the device.

Coated medical devices may also provide for the localized delivery of therapeutic agents to target locations within the body. Such localized drug delivery avoids the problems of systemic drug administration, producing unwanted effects on parts of the body that are not to be treated, or not being able to deliver a high enough concentration of therapeutic agent to the afflicted part of the body. Localized drug delivery may be achieved, for example, by coating portions of the medical devices that directly contact the inner vessel wall. This drug delivery may be intended for short and sustained periods of time.

### BRIEF DESCRIPTION

The present invention is directed to methods, processes, and systems for injecting or otherwise forcing therapeutic into porous regions of a medical device. These porous regions may be in the material comprising the medical device as well as in materials covering or otherwise masking the medical device. For example, an implantable stent may be made from a porous metallic alloy that contains numerous voids and interstices. These voids and interstices may be filled with therapeutic through high pressure and high velocity delivery methods and systems of the present invention. Likewise, an implantable stent may be coated or otherwise covered with a porous matrix that itself contains a plurality of voids and interstices. These voids or interstices may also be filled with therapeutic in accord with the present invention.

In accord with the invention, the therapeutic may be delivered and injected using an injector positioned away from the target area of the device as well as in close proximity and in contact with the target area of the medical device. The therapeutic may also be delivered in steady injections as well as in periodic bursts over uniform and non-uniform intervals. The therapeutic may still further be injected throughout the medical device as well as in specified areas or regions of the device. Moreover, the materials being injected may change during an injection cycle. For instance, a solution may be followed by a powder and then by a solid.

In each case, the voids and interstices of the porous material may be configured to control the elution rate of therapeutic and may be sized to have a mean cross-section on the order of 10⁻³ meters or smaller.

The invention may be embodied in numerous devices and through numerous methods and systems. The description provided herein, which, when taken in conjunction with the annexed drawings, discloses examples of the invention. Other embodiments, which incorporate some or all of the features and steps as taught herein, are also possible.

### BRIEF DESCRIPTION OF THE DRAWINGS

Referring to the drawings, which form a part of this disclosure:

FIG. 1 shows an injection system and a medical implant having a porous matrix region that may be employed in accord with the present invention;

FIG. 2 shows a cross-sectional view of an injector sealed against a porous matrix that may be employed in accord with the present invention;

FIG. 3a shows a porous stent comprised of a porous matrix that may be employed in accord with the present invention;

FIG. 3b shows a porous stent having a first porous matrix region and a second porous matrix region that may be employed in accord with the present invention;

FIG. 4a shows an enlarged view of a portion of the first porous matrix region of the porous stent of FIG. 3b;

FIG. 4b shows a stent having a first porous matrix region and a second porous matrix region that may be employed in accord with the present invention;

FIG. 5 shows an injection system and a plurality of stents positioned within a treatment chamber that may be employed in accord with the present invention; and

FIG. 6 is a flow chart of methods that may be employed in accord with the present invention.

### DETAILED DESCRIPTION

The present invention generally relates to injecting, driving or otherwise forcing therapeutic into one or more voids or spaces of a porous region medical device. These medical devices, which can be stents or other devices sized to be inserted into a patient, may be injected with therapeutic using methods and systems employing injectors positioned near or in contact with the devices. These injectors may be used to force therapeutic into porous regions of the medical devices. These porous regions may be resident in a porous matrix comprising the material forming the medical device and they may also comprise materials, such as coatings, placed on the medical device. The therapeutic may be driven or otherwise injected into all of the voids of the porous regions. Likewise, the therapeutic may be driven into some of the voids of the porous regions of the medical implant while not into others. In some embodiments, one type of therapeutic may be driven into a first porous region while another type of therapeutic is driven into a second porous region.

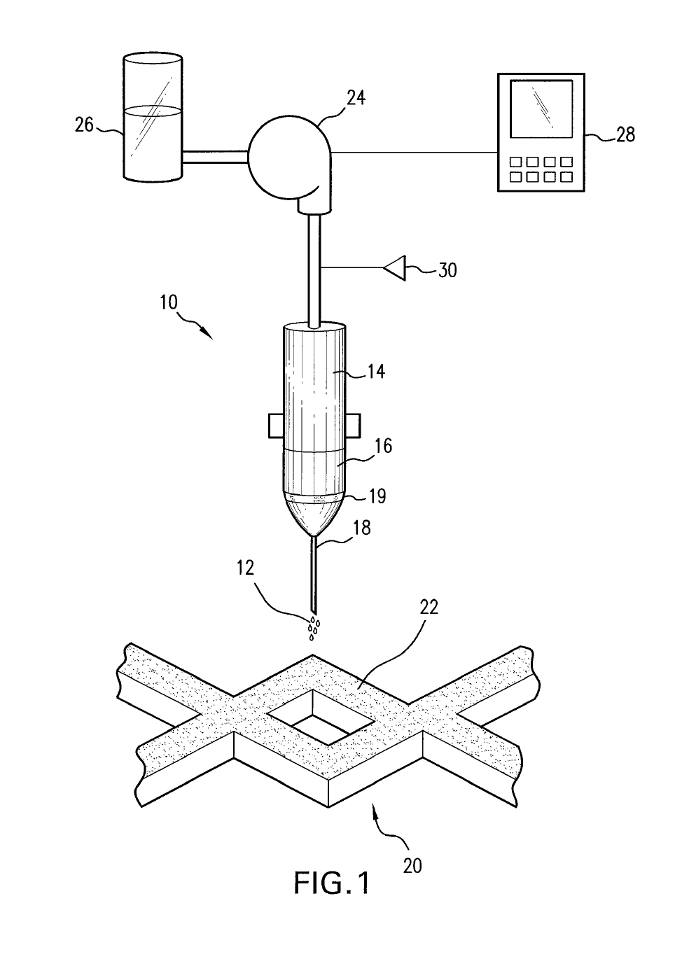

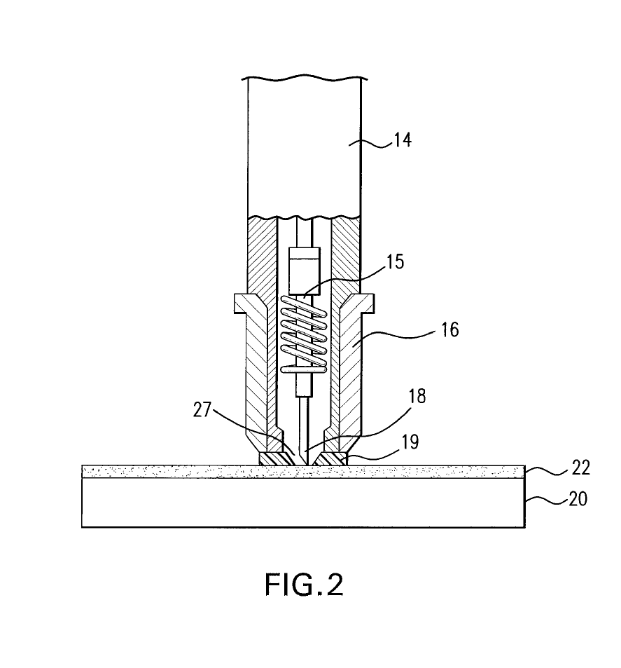

Referring initially to FIGS. 1 and 2, a high-pressure injection system 10 and a medical implant 20 having a porous matrix layer 22 deposited thereon are illustrated. The high pressure injection system 10 may be used to inject and drive therapeutic into the porous matrix layer 22 of the medical implant 20. The high pressure injection system in this figure is shown as containing an injector 14 having a nozzle 16, a seal 19, and a needle 18. The injector 14 is shown being fed by a pump 24 that is fluidly connected to a reservoir and is being controlled by a control unit 28. A pressure regulator 30 is shown positioned between the pump and the injector to sense the pressure and velocity of the therapeutic being sent to the injector by the pump and to provide this information to the controller 28 so that the controller may control the system. When therapeutic reaches the injector 14 from the pump 24, the injector may itself be configured to increase its rate of travel and the pressure under which it exits the injector 14. FIG. 2 shows that the injector 14 may contain an electronic solenoid 15, which may act to increase the speed and pressure of the therapeutic being ejected from the injector 14. Thus, the injection system 10 of FIGS. 1 and 2 may be configured to selectively drive therapeutic 12, such as polymer-free therapeutic, into the porous matrix layer 22 at high pressures and/or velocities to effectively lodge therapeutic 12 within the porous matrix layer 22.

As seen in FIGS. 1 and 2, the nozzle 16 may also include a needle 18 blocking an orifice 27. The size of the needle 18 and orifice may be tailored to supply therapeutic 12 to different regions of the medical implant 20 simultaneously.

The nozzle 16 may also include a therapeutic compatible seal 19, such as an elastomeric material. As seen in FIG. 2, the seal 19 may seal the injector 14 or exit nozzle 18 against the porous matrix layer, during some or all of the injection process, when therapeutic is ejected. This seal may also be made of soft metals, such as gold. Stainless steel, titanium or tungsten carbide may also be suitable materials. The seal may be, for example, an O-ring which also may protect the injector 14 if the injector comes into contact with the porous matrix intentionally or unintentionally. By using the seal 19, the position of the therapeutic may be limited and controlled during injections. Thus, therapeutic may be directed to areas defined by the seal of the nozzle 16.

As stated, the injector 14 may deliver therapeutic 12 to the porous matrix layer 22 at high pressures and/or velocities. For example, the therapeutic may be delivered at approximately 250 bar and at supersonic speed; other pressures and speeds may, however, also be used. For instance, the therapeutic may exit the injector 14 over ranges of pressures between about 100 bar and 2,500 bar. The size of the droplets being injected may be controlled by adjusting the injection pressures. Small droplets of therapeutic 12 may be delivered by using operating pressures between about 250-2,500 bar. Larger droplets of therapeutic may be delivered by using lower operating pressures of 100-250 bar. Higher pressures are preferred, since higher operating pressures may produce smaller droplets at much higher velocities. Smaller droplets may be preferable as they may penetrate deeply into the porous matrix 20. If larger droplets are preferred, lower pressures may be used.

The therapeutic may be injected in short bursts, cycling on and off during delivery. The therapeutic may also be injected with sustained bursts, having long injection cycle times. In each case the pressure and the velocity may be high or one of these criteria may be high while the other is not elevated. In other words, the pressure may be 1000 bar or more but the speed may be well below 500 m/s. Both high pressure pulses and continuous delivery of elevated pressures and speeds may be used to force therapeutic deep within the porous matrix layer 22.

As stated, the injection system 10 may also have a plurality of sensors, such as a pressure regulator 30, to regulate operating parameters such as the pressure and velocity at which the therapeutic 12 may be delivered. The control unit 28 may be an electronic or mechanical control system. The control unit 28 may be configured to provide different doses or quantities of therapeutic 12 via the injector 14. Further, different types of therapeutic 12 may be applied via the control unit 28 communicating with the reservoir(s) 26. The control unit 28 may also control and/or adjust the volume or “shot size” of therapeutic 12 exiting the injector 14.

The therapeutic 12 may be dispensed in solution, powder or solid form through the injector 14. Furthermore, the control unit may also regulate and change the material being injected such that a solution, powder and solid may be alternatively dispensed and injected. The therapeutic 12 may also be polymer-free to prevent tissue inflammation.

Multiple injectors 14 may be used and each injector 14 may have a nozzle 16, a reservoir 26, a pump 24, a control unit 28, and a pressure regulator 30. For example, a first injector 14 may be used to deliver one type of therapeutic 12 into a first porous region of the medical implant, while a second injector may be used to deliver another type of therapeutic 12 into a second porous region of the medical implant.

As stated, the present invention may be used with medical implants having at least one porous matrix region. The voids and interstices that comprise the porous matrix region may be various sizes, and may have dimensions in a nanometer scale and a micrometer scale. These voids and interstices may be homogenous in size and non-homogeneous in size. For example, the voids and interstices may form pores having a mean pore size of approximately 10⁻³ meters or smaller. Also, the porous matrixes may comprise material added to the device as well as the material comprising the device itself. In other words, the porous matrix region may form portions or all of the device and may also be added to the device as a coating of some kind.

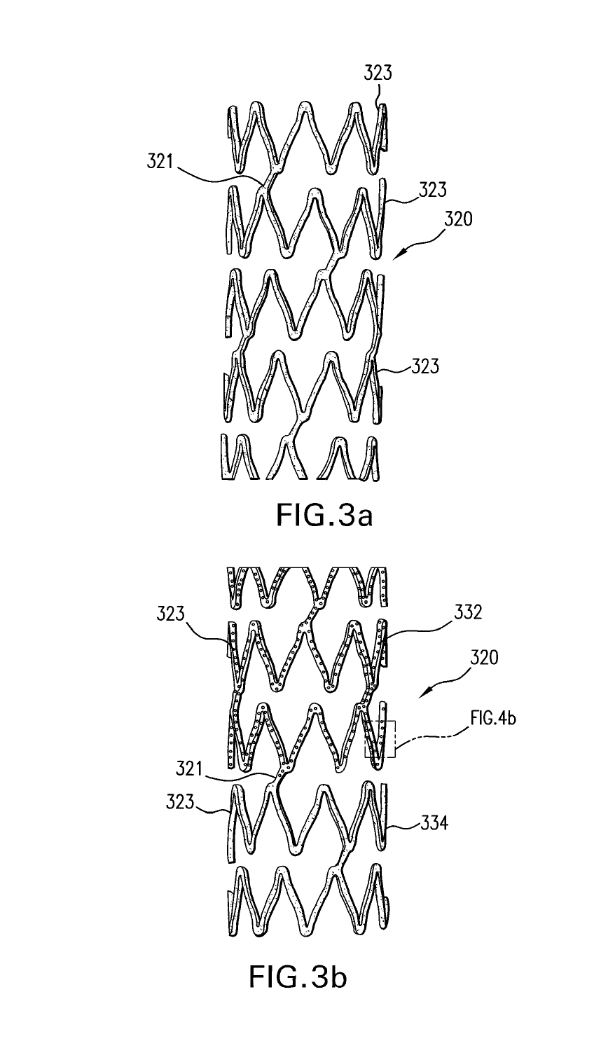

As seen in FIG. 3a, the entire stent 320 may be porous and may contain two or more porous matrix regions. For example, as seen in FIG. 3b, a stent 320 with first and second porous matrix regions 332 and 334 is provided. FIGS. 3a and 3b illustrate a stent 320 which is composed of a number of struts and links 321 made of a suitable material, such as metal, containing pores 323. In FIG. 3b, the first porous matrix region 332 may be characterized by a first porosity and first mean pore size configured to receive certain quantities and types of therapeutic while the second porous matrix region 334 shown in FIG. 3b may be characterized by a second porosity and a second mean pore size configured to receive different quantities and types of therapeutic. As noted herein above, the mean pore size may be about 10⁻³ meters or smaller. Thus, one therapeutic may be loaded into the pores 323 of the first porous matrix region 332 and a second therapeutic may be loaded into the pores 323 of the second porous matrix region 334. The same therapeutic may also be loaded into both the first and the second porous matrix regions 332, 334.

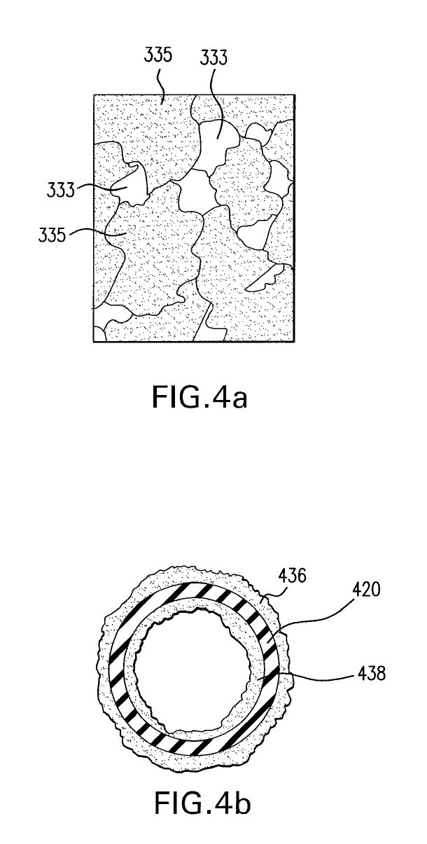

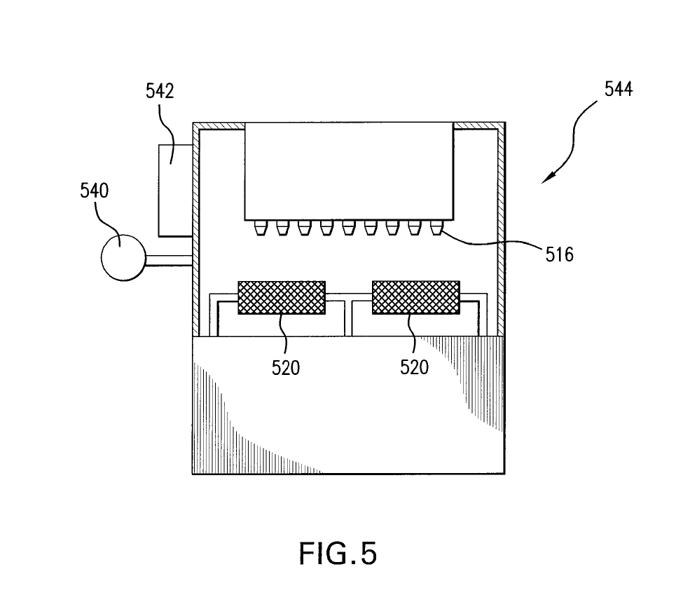

FIG. 4a shows an enlarged view of a portion of the first matrix region 332 of FIG. 3b. As can be seen, the porous matrix may include particles 335 such as carbon. The particles 335 may include pockets or pores 335 between adjacent particles 335. The proportion of the non-solid volume to the total volume of material is conventionally called the porosity of the particle material. Each pore 335 has a pore size and the rate of drug elution may be controlled by the pore size.

Since the rate of drug elution from a porous region may be determined by the pore size in the matrix, it may be preferred that the pores 335 are relatively small, for example, as stated herein, in the micro-meter or nano-meter scale. Smaller size pores 335 may enable sustained therapeutic delivery over a reasonable timescale, for example, about three months. In order to provide enough therapeutic to have a therapeutic effect, it may be preferred that all available spaces in the porous regions are loaded with therapeutic.

As stated above, instead of the medical implant being formed of a porous matrix, the medical implant may have a porous matrix layer or layers deposited thereon. For instance, as seen in FIG. 1, the stent 20 may have a porous matrix layer 22 and as seen in FIG. 4b, the stent 420 may have first and second porous matrix layers 436, 438. The first porous matrix layer 436 may be located on the outside surface of the stent 420, while the second porous matrix layer 438 may be located on the inside surface of the stent 420. Also, multiple porous matrix layers may be placed on top of one another or other surfaces of the stent may have a layer deposited thereon.

Medical implants having porous matrix regions may be made from a powdered material such as powdered metal or polymer. The medical implants of the present invention may be formed of any therapeutic-compatible powdered metals such as stainless steel. Other suitable metals include, but are not limited to, spring steel, nitinol and titanium as well as any other therapeutic-compatible metal which may become available in powdered form in the future. The porous matrix regions of these medical implants may also be prepared with different pore sizes and may be prepared having a range of porosities allowing for the production of medical implants with differing therapeutic delivery characteristics.

The medical implants in accord with the present invention may also be formed of therapeutic-compatible powdered polymeric material such as PTFE or a combination of polymeric and metal materials.

Medical implants having the porous regions described herein may be used for innumerable medical purposes, including the reinforcement of recently re-enlarged lumens, the replacement of ruptured vessels, and the treatment of disease such as vascular disease by local pharmacotherapy, i.e., delivering therapeutic drug doses to target tissues while minimizing systemic side effects. Examples of such medical implants include stents, stent grafts, vascular grafts, intraluminal paving systems, joint replacement, surgical pins, dental implants, and other devices used in connection with therapeutic or drug-loaded polymer coatings. Such medical devices are implanted or otherwise utilized in body lumina and organs such as the coronary vasculature, esophagus, trachea, colon, biliary tract, urinary tract, prostate, brain, and the like.

The medical implants themselves may be self-expanding, mechanically expandable, or hybrid implants which may have both self-expanding and mechanically expandable characteristics. The medical implant may be made in a wide variety of designs and configurations, and may be made from a variety of materials including plastics and metals. Additionally, the medical implant may be fabricated from various materials including conductive materials, such as conductive ceramic, polymeric, metallic materials.

A further step that may be employed with methods of the present invention is the step of depositing therapeutic into the porous region of the medical implant within a treatment chamber 544 via an injector system 516. A treatment chamber 544 may be made from various materials including clear, translucent, and opaque polymers, metals, and ceramics. Clear polymers, which provide for the internal viewing of implants being coated or impregnated with therapeutics in the treatment chamber 544, may be used in an exemplary embodiment.

The medical implant 520 may be rotatable within the treatment chamber 544. Furthermore, the treatment chamber may be sized to hold one or more implants. The treatment chamber may also be in fluid communication with an fluid source 540, for example, a vacuum source, to facilitate the depositing process. A compressible fluid supply source may also be plausible. The compressible fluid may be heated. A coating drying mechanism 542, such as an infrared heater or convention oven, may also be used to facilitate drying of the implant.

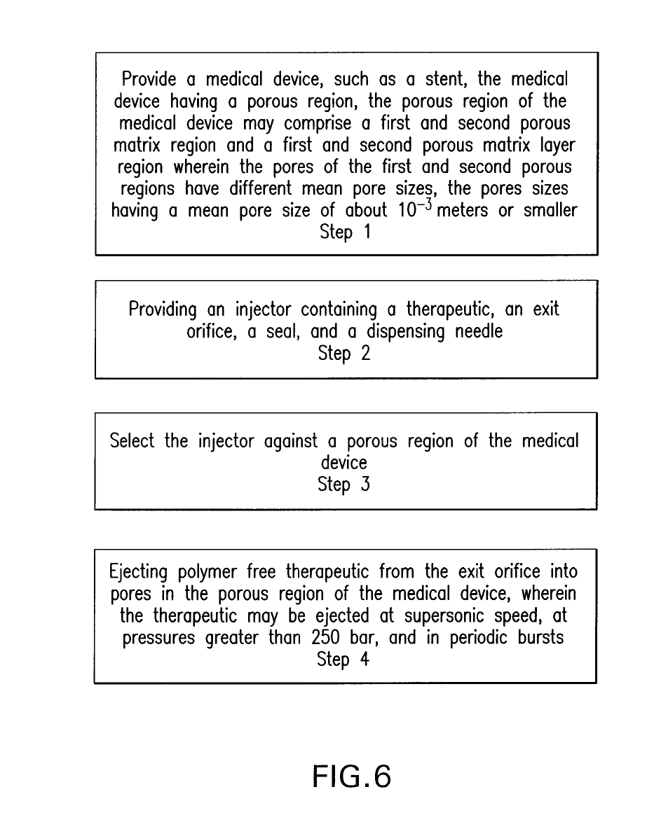

FIG. 6 shows a flow chart including method steps that may be employed with embodiments of the present invention to inject therapeutic into porous regions of a medical device. In the example of FIG. 6, step 1 may include providing a medical device, such as a stent, the medical device having a porous region, the porous region of the medical device may comprise a first and second porous matrix region and a first and second porous matrix layer region wherein the pores of the first and second porous regions have different mean pores sizes. The pore sizes having a mean pore size of about 10⁻³ meters or smaller. Step 2 may include providing an injector containing a therapeutic, an exit orifice, a seal, and a dispensing needle.

Step 3 may include sealing the injector against a porous region of the medical device. Step 4 may include ejecting polymer free therapeutic from the exit orifice into pores in the porous region of the medical device, wherein the therapeutic may be ejected at supersonic speed, at pressures greater than about 250 bar, and in periodic bursts. In alternative embodiments, not shown, the sequence of steps may be reordered and steps may be added or removed. The steps may also be modified.

While various embodiments have been described, other embodiments are plausible. It should be understood that the foregoing descriptions of various examples of the medical implant and injection system are not intended to be limiting, and any number of modifications, combinations, and alternatives of the examples may be employed to facilitate the effectiveness of depositing therapeutic into the porous matrix region and porous matrix layers.

Coatings that may be used with embodiments of the present invention, may comprise a polymeric and or therapeutic agent formed, for example, by admixing a drug agent with a liquid polymer, in the absence of a solvent, to form a liquid polymer/drug agent mixture. A suitable list of drugs and/or polymer combinations is listed below. The term “therapeutic agent” as used herein includes one or more “therapeutic agents” or “drugs.” The terms “therapeutic agents” or “drugs” can be used interchangeably herein and include pharmaceutically active compounds, nucleic acids with and without carrier vectors such as lipids, compacting agents (such as histones), viruses (such as adenovirus, adenoassociated virus, retrovirus, lentivirus and α-virus), polymers, hyaluronic acid, proteins, cells and the like, with or without targeting sequences.

Specific examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promoters such as growth factors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and β, platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMPs”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMPs are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNAs encoding them.

Coatings used with embodiments of the present invention may comprise a polymeric material/drug agent matrix formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof. Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

The polymer used in the exemplary embodiments of the present invention is preferably capable of absorbing a substantial amount of drug solution. When applied as a coating on a medical device in accordance with the present invention, the dry polymer is typically on the order of from about 1 to about 50 microns thick. In the case of a balloon catheter, the thickness is preferably about 1 to 10 microns thick, and more preferably about 2 to 5 microns. Very thin polymer coatings, e.g., of about 0.2-0.3 microns and much thicker coatings, e.g., more than 10 microns, are also possible. It is also within the scope of the present invention to apply multiple layers of polymer coating onto a medical device. Such multiple layers are of the same or different polymer materials.

The polymer of the present invention may be hydrophilic or hydrophobic, and may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers.

Coatings from polymer dispersions such as polyurethane dispersions (BAYHYDROL®, etc.) and acrylic latex dispersions may also be used with the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In another preferred embodiment, the polymer is a copolymer of polylactic acid and polycaprolactone.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. A method of injecting therapeutic into pores of a medical device, the method comprising:
providing a medical device sized to be inserted into a patient, the medical device having at least a portion thereof comprising a porous region, the porous region having a plurality of pores sized with a mean pore size of 10⁻³ meters or smaller;
providing an injector containing a therapeutic and having an exit orifice;
positioning the exit nozzle to be in fluid communication with the medical device; and
ejecting therapeutic from the exit orifice of the injector and into a plurality of the pores in the porous region of the medical device,
wherein the therapeutic is ejected at pressures greater than about 100 bar.

2. The method of claim 1, wherein the injector is sealed against the porous region when the therapeutic is ejected from the injector.

3. The method of claim 1, wherein the therapeutic is ejected from the exit orifice at supersonic speed and pressures greater than 250 bar.

4. The method of claim 1, wherein the therapeutic is polymer-free.

5. The method of claim 2, wherein the injector contains a seal positioned to seal against the porous region when therapeutic is ejected from the exit nozzle.

6. The method of claim 1, wherein the injector comprises a dispensing needle.

7. The method of claim 1, wherein the therapeutic is ejected from the exit orifice in periodic bursts over a period of time.

8. The method of claim 1, wherein the medical device is a medical implant.

9. The method of claim 1, wherein the medical device is a stent.

10. The method of claim 1, wherein the porous region is a porous matrix region comprises the medical device.

11. The method of claim 1, wherein the porous region is a porous matrix layer positioned on the medical device.

12. The method of claim 1 wherein the porous region comprises a first porous matrix layer and a second porous matrix layer.

13. The method of claim 1 wherein the porous region comprises a first porous matrix region and a second porous matrix region.

14. The method of claim 13 wherein the pores of the first porous matrix region have a first mean pore size and the pores of the second porous matrix region have a second mean pore size.
